(19) United States
(12) Patent Application Publication
(10) Pub. No.: US 2007/0016058 A1
(43) Pub. Date: Jan. 18, 2007

(54) System and method for controlling ultrasound probe having multiple transducer arrays

(76) Inventor: Scott Kerwin, Charlotte, NC (US)

Correspondence Address: THE SMALL PATENT LAW GROUP LLP, 611 OLIVE STREET, SUITE 1611, ST. LOUIS, MO, 63101, US

(21) Appl. No.: 11/182,475

(22) Filed: Jul. 15, 2005

(51) Int. Cl. A61B 8/14

(52) U.S. Cl. 600/459

(57) ABSTRACT

A system and method for controlling an ultrasound probe are provided. The ultrasound probe includes a first transducer array and a second transducer array. The ultrasound probe further includes a transducer array selector component within a housing. The transducer array selector component is configured to generate a control signal based on a user input to selectively activate one of the first transducer array and the second transducer array.

### BACKGROUND OF THE INVENTION

This invention relates generally to ultrasound systems and, more particularly, to probes for ultrasound medical imaging systems.

Ultrasound systems typically include ultrasound scanning devices, such as, ultrasound probes having different transducers that allow for performing various different ultrasound scans (e.g., different imaging of a volume or body). The ultrasound probes are typically connected to an ultrasound system for controlling the operation of the probes. The probes include a scan head having a plurality of transducer elements (e.g., piezoelectric crystals), which may be arranged in one or more arrays. The ultrasound system drives the transducer elements within the array during operation, such as, during a scan of a volume or body, which may be controlled based upon the type of scan to be performed. The ultrasound system includes a plurality of channels for communicating with the probe. For example, the channels may transmit pulses for driving the transducer elements and for receiving signals therefrom.

In transrectal probes, two separate connectors are used to connect the probe to the ultrasound system controlling the probe. Each of these connectors corresponds to a transducer array of the ultrasound probe. Specifically, one connector is used to control a transverse transducer array of the ultrasound probe and the other connector is used to control a longitudinal transducer array of the ultrasound probe. A user must switch between transducer arrays using the ultrasound system.

With this connection arrangement, an individual connecting the ultrasound probe to the ultrasound system must be careful to make the proper connections. If the connections are reversed, the system will not properly operate. Further, a user performing a scan with the this connection arrangement must access and operate the ultrasound system in order to switch between the transducer arrays (e.g., turn around and access the ultrasound controller).

Thus, it not only may be difficult to operate these ultrasound systems because a user has to, for example, turn around to reach the ultrasound system control panel in order to switch between transducer arrays, but the connectors must be properly identified to allow for proper connection of the ultrasound probe to the ultrasound system. Further, the use of two connectors and corresponding cables not only adds complexity to the ultrasound system, but also adds cost.

### BRIEF DESCRIPTION OF THE INVENTION

In an exemplary embodiment, an ultrasound probe is provided that includes a first transducer array and a second transducer array. The ultrasound probe further includes a transducer array selector component within a housing. The transducer array selector component is configured to generate a control signal based on a user input to selectively activate one of the first transducer array and the second transducer array.

In another exemplary embodiment, an ultrasound probe is provided that includes a first transducer array, a second transducer array and a single system cable for connecting to an ultrasound system. The ultrasound probe further includes a user input device configured to receive a user input and a transducer array selector component within a housing. The transducer array selector component is configured to generate a control signal based on the received user input, with the control signal communicated to the ultrasound system via the single system cable for selectively activating one of the first transducer array and the second transducer array.

In yet another exemplary embodiment, a method for controlling an ultrasound probe is provided. The method includes generating a control signal based on a user input and selectively activating one of a first transducer array and a second transducer array based on the control signal.

### BRIEF DESCRIPTION OF THE DRAWINGS

FIG. 1 is a block diagram of an ultrasound system in accordance with an exemplary embodiment of the present invention.

FIG. 2 is a block diagram of an ultrasound system in accordance with another exemplary embodiment of the present invention.

FIG. 3 is a perspective view of an image of an object acquired by the systems of FIGS. 1 and 2 in accordance with an exemplary embodiment of the present invention.

FIG. 4 is a block diagram of an ultrasound probe in communication with a host system in accordance with an exemplary embodiment of the present invention.

FIG. 5 is a block diagram showing a multiplexing arrangement in accordance with an exemplary embodiment of the present invention.

FIG. 6 is a schematic diagram of a transducer array selector component in accordance with an exemplary embodiment of the present invention.

FIG. 7 is a diagram of an ultrasound probe having a user input device in accordance with an exemplary embodiment of the invention.

### DETAILED DESCRIPTION OF THE INVENTION

Exemplary embodiments of ultrasound systems and methods for controlling ultrasound probes are described in detail below. In particular, a detailed description of exemplary ultrasound systems will first be provided followed by a detailed description of various embodiments of methods and systems for controlling ultrasound probes having multiple transducer arrays.

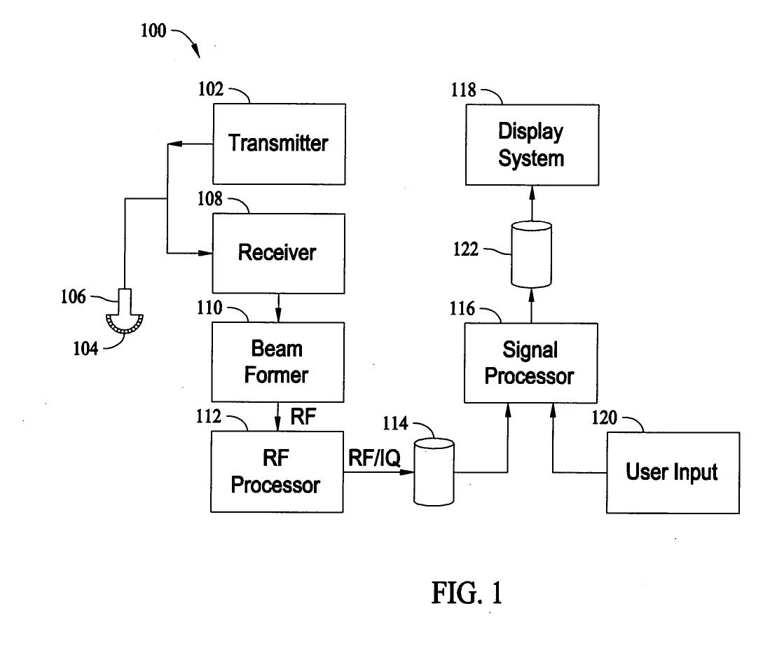

FIG. 1 illustrates a block diagram of an exemplary embodiment of an ultrasound system 100 that may be used, for example, to acquire and process ultrasonic images. The ultrasound system 100 includes a transmitter 102 that drives one or more arrays of elements 104 (e.g., piezoelectric crystals) within or formed as part of one or more transducers 106 to emit pulsed ultrasonic signals into a body or volume. A variety of geometries may be used and one or more transducers 106 may be provided as part of a probe (not shown) as described in more detail herein. The pulsed ultrasonic signals are back-scattered from density interfaces and/or structures, for example, in a body, like blood cells or muscular tissue, to produce echoes that return to the elements 104. The echoes are received by a receiver 108 and provided to a beamformer 110. The beamformer performs beamforming on the received echoes and outputs an RF signal. The RF signal is then processed by an RF processor 112. The RF processor 112 may include a complex demodulator (not shown) that demodulates the RF signal to form IQ data pairs representative of the echo signals. The RF or IQ signal data then may be routed directly to an RF/IQ buffer 114 for storage (e.g., temporary storage).

The ultrasound system 100 also includes a signal processor 116 to process the acquired ultrasound information (i.e., RF signal data or IQ data pairs) and prepare frames of ultrasound information for display on a display system 118. The signal processor 116 is adapted to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 114 during a scanning session and processed in less than real-time in a live or off-line operation.

The ultrasound system 100 may continuously acquire ultrasound information at a frame rate that exceeds fifty frames per second, which is the approximate perception rate of the human eye. The acquired ultrasound information is displayed on the display system 118 at a slower frame-rate. An image buffer 122 may be included for storing processed frames of acquired ultrasound information that are not scheduled to be displayed immediately. In an exemplary embodiment, the image buffer 122 is of sufficient capacity to store at least several seconds of frames of ultrasound information. The frames of ultrasound information may be stored in a manner to facilitate retrieval thereof according to their order or time of acquisition. The image buffer 122 may comprise any known data storage medium.

A user input device 120 may be used to control operation of the ultrasound system 100. The user input device 120 may be any suitable device and/or user interface for receiving user inputs to control, for example, the type of scan or type of transducer to be used in a scan.

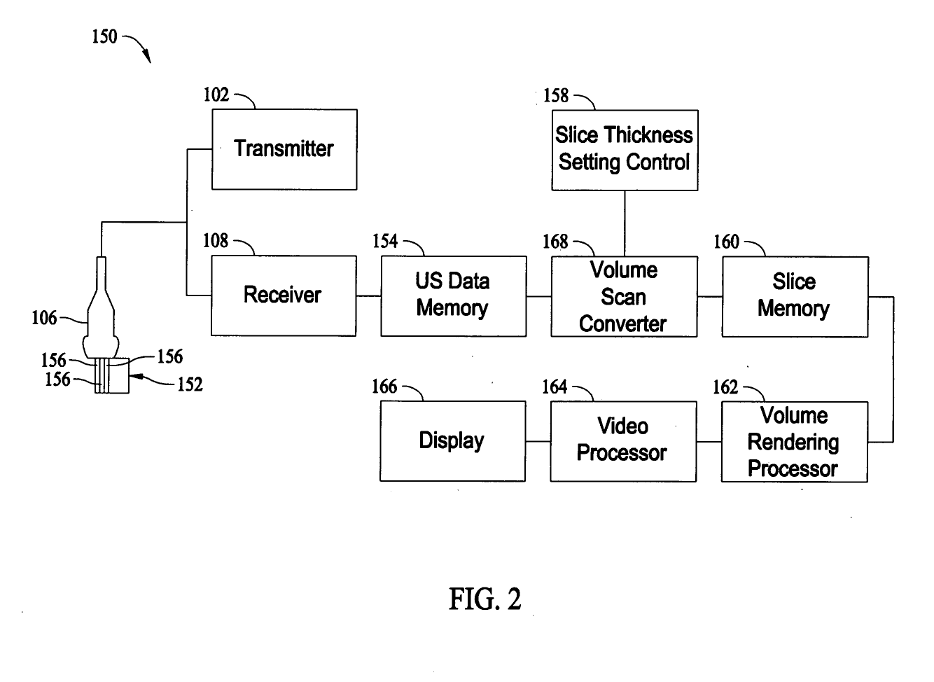

FIG. 2 illustrates a block diagram of another exemplary embodiment of an ultrasound system 150 that may be used, for example, to acquire and process ultrasonic images. The ultrasound system 150 includes one or more transducers 106 in communication with the transmitter 102 and receiver 108. The transducer(s) 106 transmit ultrasonic pulses and receives echoes from structures inside a scanned ultrasound volume 152. A memory 154 stores ultrasound data from the receiver 108 derived from the scanned ultrasound volume 152. The scanned ultrasound volume 152 may be obtained by various techniques, including, for example, 3D scanning, real-time 3D imaging, volume scanning, scanning with transducers having positioning sensors, freehand scanning using a voxel correlation technique, 2D scanning or scanning with a matrix of array transducers, among others.

The transducer 106 is moved, such as along a linear or arcuate path, while scanning a region of interest (ROI). At each linear or arcuate position, the transducer 106 obtains a plurality of scan planes 156. The scan planes 156 are collected for a thickness, such as from a group or set of adjacent scan planes 156. The scan planes 156 are stored in the memory 154, and then provided to a volume scan converter 168. In some exemplary embodiments, the transducer 106 may obtain lines instead of the scan planes 156, with the memory 154 storing lines obtained by the transducer 106 rather than the scan planes 156. The volume scan converter 168 receives a slice thickness setting from a slice thickness setting control 158, which identifies the thickness of a slice to be created from the scan planes 156. The volume scan converter 168 creates a data slice from multiple adjacent scan planes 156. The number of adjacent scan planes 156 that are obtained to form each data slice is dependent upon the thickness selected by the slice thickness setting control 158. The data slice is stored in a slice memory 160 and accessed by a volume rendering processor 162. The volume rendering processor 162 performs volume rendering upon the data slice. The output of the volume rendering processor 162 is provided to a video processor 164 that processes the volume rendered data slice for display on a display 166.

It should be noted that the position of each echo signal sample (Voxel) is defined in terms of geometrical accuracy (i.e., the distance from one Voxel to the next) and one or more ultrasonic responses (and derived values from the ultrasonic response). Suitable ultrasonic responses include gray scale values, color flow values, and angio or power Doppler information. It should be noted that the ultrasound system 150 also may include a user input or user interface for controlling the operation of the ultrasound system 150.

It should further be noted that the ultrasound systems 100 and 150 may include additional or different components. For example, the ultrasound system 150 may include a user interface or user input 120 (shown in FIG. 1) to control the operation of the ultrasound system 150, including, to control the input of patient data, scan parameters, a change of scan mode, and the like. Further, various embodiments of the present invention, and as described in more detail herein, provide a user interface, input or control as part of an ultrasound probe.

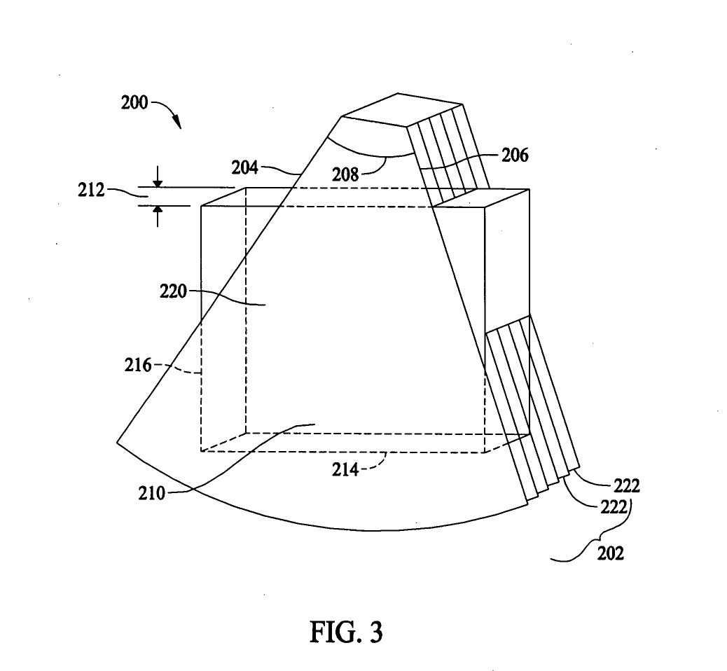

FIG. 3 illustrates an exemplary image of an object 200 that may be acquired by the ultrasound systems 100 and 150. The object 200 includes a volume 202 defined by a plurality of sector shaped cross-sections with radial borders 204 and 206 diverging from one another at an angle 208. The transducer 106 (shown in FIGS. 1 and 2) electronically focuses and directs ultrasound firings longitudinally to scan along adjacent scan lines in each scan plane 156 (shown in FIG. 2) and electronically or mechanically focuses and directs ultrasound firings laterally to scan adjacent scan planes 156. The scan planes 156 obtained by the transducer 106, and as illustrated in FIG. 1, are stored in the memory 154 and are scan converted from spherical to Cartesian coordinates by the volume scan converter 168. A volume comprising multiple scan planes 156 is output from the volume scan converter 168 and stored in the slice memory 160 as a rendering region 210. The rendering region 210 in the slice memory 160 is formed from multiple adjacent scan planes 156.

The rendering region 210 may be defined in size by an operator using a user interface or input to have a slice thickness 212, width 214 and height 216. The volume scan converter 168 (shown in FIG. 2) may be controlled by the slice thickness setting control 158 (shown in FIG. 2) to adjust the thickness parameter of the slice to form a rendering region 210 of the desired thickness. The rendering region 210 defines the portion of the scanned ultrasound volume 152 that is volume rendered. The volume rendering processor 162 accesses the slice memory 160 and renders along the slice thickness 212 of the rendering region 210.

Referring now to FIGS. 1 and 2, during operation, a slice having a pre-defined, substantially constant thickness (also referred to as the rendering region 210) is determined by the slice thickness setting control 158 and is processed in the volume scan converter 168. The echo data representing the rendering region 210 (shown in FIG. 3) may be stored in the slice memory 160. Predefined thicknesses between about 2 mm and about 20 mm are typical, however, thicknesses less than about 2 mm or greater than about 20 mm may also be suitable depending on the application and the size of the area to be scanned. The slice thickness setting control 158 may include a control member, such as a rotatable knob with discrete or continuous thickness settings.

The volume rendering processor 162 projects the rendering region 210 onto an image portion 220 of an image plane(s) 222 (shown in FIG. 3). Following processing in the volume rendering processor 162, pixel data in the image portion 220 may be processed by the video processor 164 and then displayed on the display 166. The rendering region 210 may be located at any position and oriented at any direction within the volume 202. In some situations, depending on the size of the region being scanned, it may be advantageous for the rendering region 210 to be only a small portion of the volume 202.

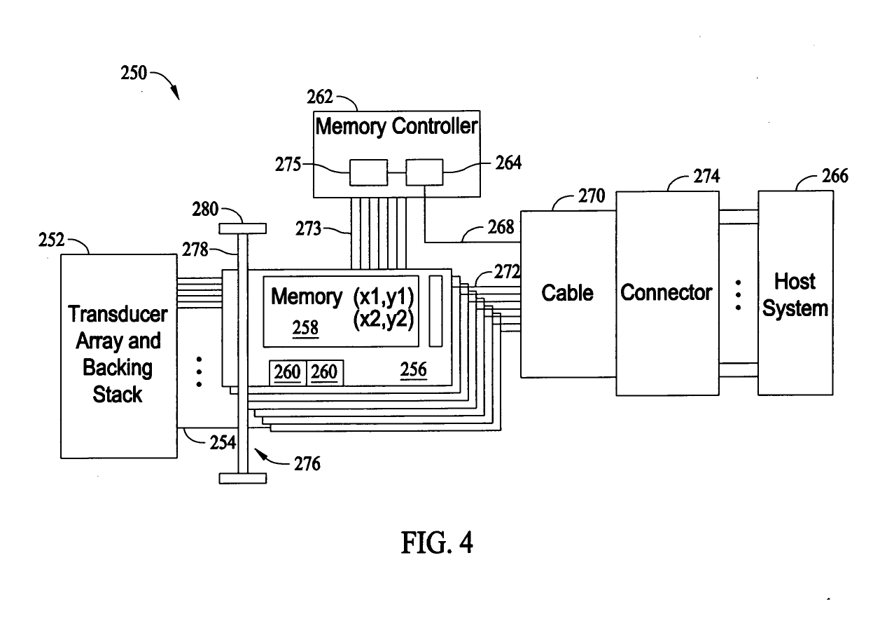

FIG. 4 illustrates a block diagram of an exemplary embodiment of an ultrasound probe 250, for example, a transrectal probe, that may be used in connection with the ultrasound systems 100 or 150. The ultrasound probe 250 includes a transducer array and backing stack 252 (the “transducer array 252”), communication cables, for example, transducer flex cables 254, which may be formed as a scan head cable, and multiple processing boards 256 that support processing electronics. Each processing board 256 may includes a location memory 258 (which may include geometry RAM, encoder RAM, location registers and control registers as noted below) and signal processors 260. A location memory controller 262 (e.g., a general purpose CPU, microcontroller, PLD, or the like) also may be provided and includes a communication interface 264.

It should be noted that one or more transducer arrays 252 may be provided. For example, in a transrectal probe constructed in accordance with an exemplary embodiment of the present invention, a transverse transducer array and a longitudinal transducer array are both provided and described in more detail herein. Further, and as described in more detail herein, multiplexing circuits for separately controlling each of the transducer arrays are provided in accordance with various embodiments of the invention.

Referring again to FIG. 4, the communication interface 264 establishes data exchange with a host system 266 over communication lines 268 (e.g., digital signal lines) and through a system cable 270. The system cable 270 may include, for example, a coaxial cable that connects to the processing boards 256 to communicate transmit pulse waveforms to the transducer array(s) 252 and communicate receive signals, after beamforming, to the host system 266. The probe 250 also may include a connector 274, through which the probe 250 connects to the host system 266.

A clamp 276 may be provided to hold the transducer flex cables 254 against the processing boards 256. The clamp 276 thereby aids in establishing electrical connectivity between the transducer flex cables 254 and the processing boards 256. The clamp 276 may include a dowel pin 278 and a bolt 280, although other implementations are also suitable.

The transducer array 252 may be bonded onto a backing stack. Further, the transducer flex cables 254 provide electrical signal connections through the backing stack. Additionally, the processing boards 256 may, like the flex cables 254, be formed from a flex material, such as, for example, polyimide, polyester, etc. The processing boards 256 may include the processing electronics for the transducer array 252, including the signal processors 260 that perform beamforming on receive apertures in the transducer array 252, as well as multiplexing circuits and switches for controlling the elements of the transducer array 252.

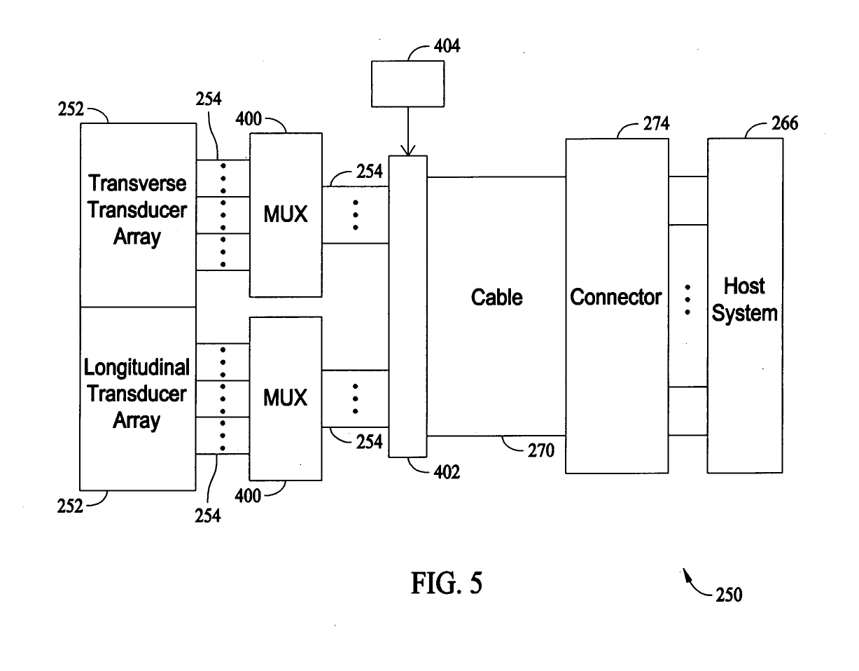

Various embodiments of the present invention include one or more signal control circuits for controlling the communication of signals between the host system 266 (shown in FIG. 4) and transducer array 252 (shown in FIG. 4). In one exemplary embodiment as shown in FIG. 5, the one or more signal control circuits include one or more multiplexing circuits 400 having connected thereto control lines, for example, the transducer flex cables 254 from the transducer array 252 for multiplexing signals between the transducer array 252 and the host system 266. For example, a printed circuit board having surface mounted integrated circuits housing switches therein (e.g., MOSFETs) may be used to control the switching of each of the transducer arrays 252, and more specifically, the connection of transducer elements to one or more channels of the ultrasound system 100 or 150 (e.g., connected to one or more channels of the host system 266 (shown in FIG. 4)). Specifically, each of the multiplexing circuits 400 control the transmission of signal pulses to their corresponding transducer array 252 that drive the transducer elements, such as, for example, piezoelectric ceramic. The multiplexing circuits 400 also control the communication of ultrasound signals received by the piezoelectric ceramics that are communicated to the host system 266.

In the exemplary embodiment shown in FIG. 5, two transducer arrays 252 (e.g., a first and second transducer array) are provided, each controlled by a separate multiplexing circuit 400. For example, the ultrasound probe 250 may be a transrectal probe having a transverse transducer array 252 and a longitudinal transducer array 252 each controlled by a corresponding multiplexing circuit 400. Further, as described in more detail herein, a transducer array controller, and more particularly, a transducer array selector component 402 is configured to generate one or more control signals to activate one of the multiplexing circuits 400 to thereby control a corresponding one of the transducer arrays 252. Additionally, and as described in more detail herein, a user input device 404, for example, a depressible button or switch, may be provided for activation by a user of the ultrasound probe 250 to selectively activate one of the transducer arrays 252.

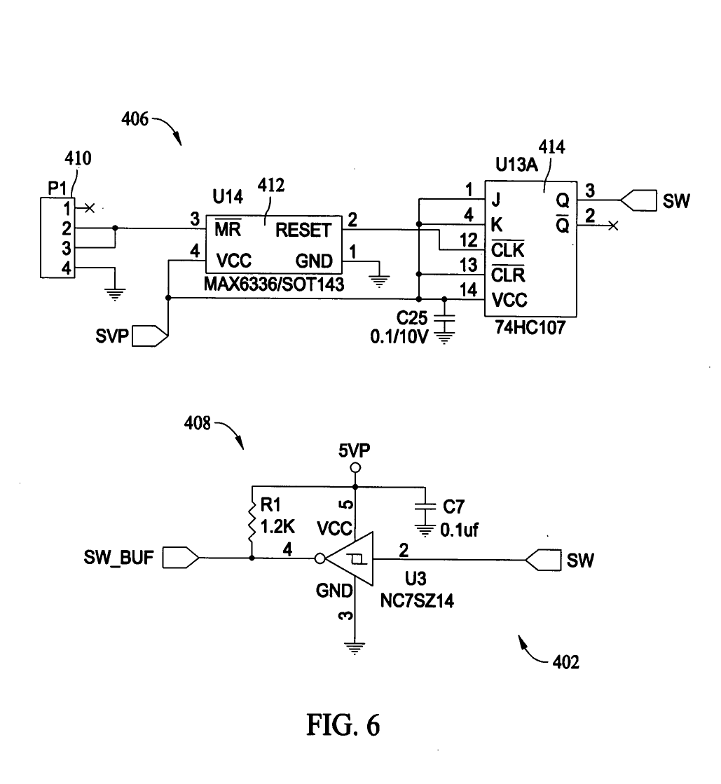

An exemplary embodiment of the transducer array selector component 402 is shown in FIG. 6. In general, the transducer array selector component 402 includes a switching circuit 406 and a buffer circuit 408. The switching circuit 406 receives a signal from the user input 404, for example, a signal responsive to a user depressing a button. In particular, a switch input receiver 410 receives a signal responsive to a user input. The input signal is filtered using a debounce circuit 412, as is known, and then provided to a switching component, for example, a flip flop, such as a JK flip flop 414. Essentially, the received signal from the user input 404 toggles the JK flip flop 414 to output either a logic high signal or a logic low signal. These logic signals are used to select one of the two multiplexing circuits 400. For example, a logic 0 (low) may correspond to the multiplexing circuit 400 for the longitudinal transducer array 252 (both shown in FIG. 5) and a logic 1 (high) may correspond the multiplexing circuit 400 for the transverse transducer array 252 (both shown in FIG. 5).

The output of the JK flip flop 414 is provided to the buffer circuit 408 that outputs a buffered signal to the system cable 270 as is known. For example, based on the type of ultrasound system to which the ultrasound probe 250 is connected, the control signals may need to be attenuated before transmitting to the ultrasound system.

In operation the transducer array selector component 402 receives an input from, for example, a button, that then toggles a switch to generate a control signal that is communicated through the system cable 270 to a connected ultrasound system to indicate which transducer array 252 to control using the corresponding multiplexing circuit 400. For example, a user may depress a button to select between a transverse transducer array 252 and a longitudinal transducer array 252 of a transrectal probe. Essentially, upon receiving a control signal from the transducer array selector component 402, the ultrasound system activates the multiplexing circuit 400 for the transducer array 252 based on the control signal.

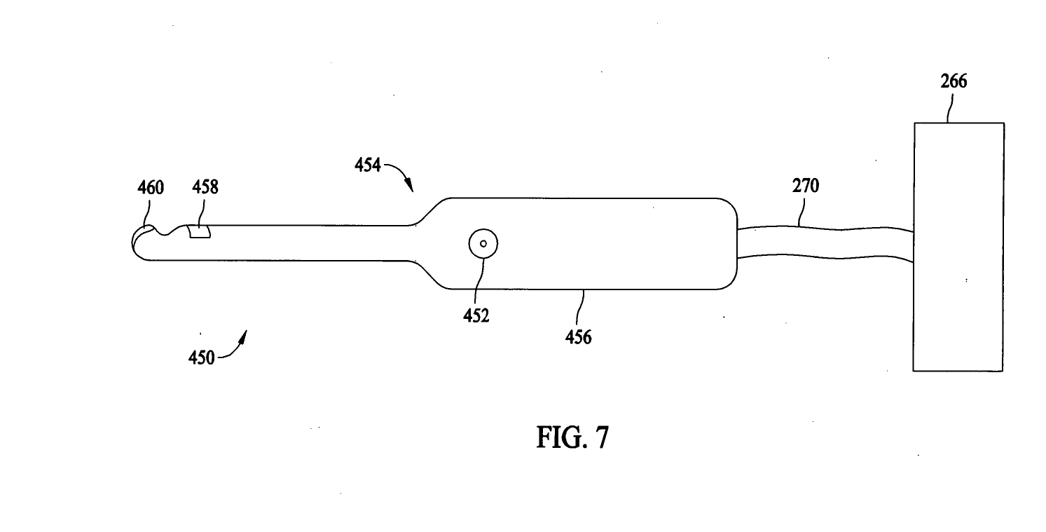

Thus, as shown in FIG. 7, a transrectal probe 450 may be provided with a user input, such as a button 452 on a housing 454, and more particularly, on a handle 456 of the transrectal probe 450. The button 452, may be, for example, a duraswitch type button, and wherein activation of the button 452 by depressing the button 452 switches between a transverse transducer array 458 and a longitudinal transducer array 460. As described herein, the activation of the button 452 causes a control signal to be communicated to the host system 266 via a single system cable 270 from the transrectal probe 450 and connected thereto. Thus, a user can select a transducer array using an input on the transrectal probe 450 without having to access the host system 266. It should be noted that in an exemplary embodiment, the transducer array selector component 402 is provided within the housing 454.

It should further be noted that the various embodiments for controlling selection of a transducer array of an ultrasound probe may be implemented without the use of a user input on the ultrasound probe. For example, in another exemplary embodiment, a selector switch or display selection on the host system 266, for example, on a display of an ultrasound controller, may include a selector for selecting one of the transducer arrays. Operation of the control functionality after selecting a transducer array is provided as described herein.

Further, it should be noted that additional components or modifications may be implemented to the various embodiments. For example, a shielded layer may be provided as part of the button 452 in any known manner to shield the ultrasound probe from electromagnetic interference (EMI) noise generated by the button 452 or other user input.

While the invention has been described in terms of various specific embodiments, those skilled in the art will recognize that the invention can be practiced with modification within the spirit and scope of the claims.

What is claimed is:

1. An ultrasound probe comprising:
a first transducer array;
a second transducer array; and
a transducer array selector component within a housing and configured to generate a control signal based on a user input to selectively activate one of the first transducer array and the second transducer array.

2. An ultrasound probe in accordance with claim 1 further comprising a user input device for activating the transducer array selector component.

3. An ultrasound probe in accordance with claim 2 wherein the user input device is provided as part of the housing.

4. An ultrasound probe in accordance with claim 2 wherein the user input device comprises a user depressible button on the housing.

5. An ultrasound probe in accordance with claim 2 wherein the user input device further comprises an electromagnetic interference (EMI) shield.

6. An ultrasound probe in accordance with claim 1 wherein the transducer array selector component is configured to generate a control signal to activate one of the first and second transducer arrays.

7. An ultrasound probe in accordance with claim 1 wherein the first transducer array comprises a transverse transducer array and the second transducer array comprises a longitudinal transducer array.

8. An ultrasound probe in accordance with claim 1 wherein the transducer array selector component is configured to generate a control signal to select between multiplexing circuits corresponding to each of the first and second transducer arrays to selectively control the corresponding one of the first and second transducer arrays.

9. An ultrasound probe in accordance with claim 1 further comprising a single system cable configured to connect to an ultrasound system.

10. An ultrasound probe in accordance with claim 1 wherein the first and second transducer arrays are configured to provide transrectal scanning.

11. An ultrasound probe comprising:
a first transducer array;
a second transducer array;
a single system cable for connecting to an ultrasound system;
a user input device configured to receive a user input; and
a transducer array selector component within a housing and configured to generate a control signal based on the received user input, the control signal communicated to the ultrasound system via the single system cable for selectively activating one of the first transducer array and the second transducer array.

12. An ultrasound probe in accordance with claim 11 wherein the user input device comprise a user depressible button.

13. An ultrasound probe in accordance with claim 11 wherein the user input device is provided as part of the housing.

14. An ultrasound probe in accordance with claim 11 further comprising a multiplexing circuit corresponding to each of the transducer arrays and selectively activated by the control signal.

15. An ultrasound probe in accordance with claim 11 wherein the first transducer array comprises a transverse transducer array and the second transducer array comprises a longitudinal transducer array.

16. An ultrasound probe in accordance with claim 11 further comprising a multiplexing circuit in the housing and corresponding to each of the first and second transducer arrays, the control signal selecting one of the multiplexing circuits for operation based on the received user input.

17. An ultrasound probe in accordance with claim 11 wherein the transducer array selector component comprises a flip flop for toggling the control signal.

18. A method for controlling an ultrasound probe, the method comprising:
generating a control signal based on a user input; and
selectively activating one of a first transducer array and a second transducer array based on the control signal.

19. A method in accordance with claim 18 further comprising receiving a control signal at the ultrasound probe.

20. A method in accordance with claim 18 wherein the control signal is generated based on a user activation of a user input device.
